(19) United States
(12) Patent Application Publication
(10) Pub. No.: US 2023/0142873 A1
(43) Pub. Date: May 11, 2023

(54) BACTERIUM OF THE CHRISTENSENELLACEAE FAMILY AND COMPOSITION CONTAINING SAME FOR PREVENTING AND/OR TREATING RENAL INSUFFICIENCY

(71) Applicant: YSOPIA BIOSCIENCES, BORDEAUX CEDEX (FR)

(72) Inventors: Georges RAWADI, PARIS (FR); Sandrine CLAUS, BORDEAUX (FR); Laure RINALDI, BORDEAUX (FR)

(21) Appl. No.: 17/915,620

(22) PCT Filed: Jun. 24, 2020
(86) PCT No.: PCT/EP2020/067630

§ 371 Date: Sep. 29, 2022

(30) Foreign Application Priority Data
Jun. 25, 2019 (FR) FR1906842

(51) Int. Cl. A61K 35/74; A61K 45/06; A61P 13/12

(52) U.S. Cl. CPC A61K 35/74; A61K 45/06; A61P 13/12; A61K 2035/115

(57) ABSTRACT

The invention relates to bacteria of the Christensenellaceae family and compositions containing same for their use in the prevention and/or treatment of renal insufficiency in humans or animals.

### TECHNICAL FIELD

The invention relates to the prevention and treatment of renal insufficiency. In particular, the invention relates to bacteria specific to the gut microbiota and to compositions containing them for preventing and/or treating renal insufficiency.

### PRIOR ART

Renal insufficiency is a disease whose pathophysiology is directly related to the destruction of kidney cells. This destruction of kidney cells leading to renal insufficiency is caused by nephropathy. There are different types of nephropathy, such as in particular vascular and hypertensive nephropathy, diabetic nephropathy mainly with type 2 diabetes, chronic glomerulonephritis, hereditary nephropathy, mainly autosomal dominant polycystic kidney disease, chronic interstitial nephropathy, various nephropathies, and nephropathies of undetermined origin.

Chronic renal insufficiency affects a large portion of the population. The health consequences are major, as it is a debilitating, painful pathology associated with a high risk of early mortality.

Currently, the treatment of chronic renal insufficiency is based on:
managing the disease at the origin of the renal insufficiency (arterial hypertension, diabetes, polycystosis, etc.), which is often at a very advanced stage when chronic renal insufficiency appears,
   slowing the evolution toward chronic renal insufficiency by protecting the kidneys,
   interventions intended to slow the progression of chronic kidney disease, in particular through strict control of blood pressure, reducing proteinuria, using converting enzyme inhibitors or angiotensin II type 1 receptor blockers, preventing episodes of acute renal failure and nephrotoxicity, moderate protein restriction adapted to the patient, controlling diabetes if it exists and stopping smoking,
   dialysis and kidney transplantation if end-stage renal failure is reached.

However, these different treatments have highly variable response rates, in particular due to the large number of factors involved in the onset of renal insufficiency, in particular chronic renal insufficiency, and due to the degradation of molecules when they are administered. In addition, these treatments have significant side effects such as: headaches, nausea, fatigue, hypoglycemia, water and sodium retention, digestive side effects (flatulence), hypotension, loss of muscle, hyperkalemia, angioedema, fetotoxicity, cramps, sleep disorders, itching, stenosis, thrombosis, lymphocele, hematoma, acute tubulonephritis, transplant rejection, diabetes, increased risk of cancer, risk of cardiovascular disease, liver disease.

Thus, there is a great need for an effective treatment of renal insufficiency, in particular chronic renal insufficiency, that works whatever the factor of origin of the disease, that is easy to administer, and that does not present side effects.

### SUMMARY OF THE INVENTION

This is the objective of the present invention, which, to respond to this need, focuses on the use of particular bacteria of the human gut microbiota, namely bacteria of the Christensenellaceae family.

Bacteria of the Christensenellaceae family, including the genus Christensenella, have already been studied and described. This is the case in particular for Christensenella minuta, Christensenella massiliensis and Christensenella timonensis. Christensenella minuta in particular was described for the first time in 2012. In 2014, a study showed that it was the most heritable taxon in a cohort of British twins and that its presence is associated with a low body mass index. This correlation between Christensenella minuta and low body mass index was then observed in a dozen studies published since 2014 in geographically diverse populations.

Surprisingly, according to the invention, the bacteria of the Christensenellaceae family, when they are administered to humans or to animals, are capable of acting on the markers of renal insufficiency in order to treat this disease.

Therefore, the subject of the invention is a bacterium of the Christensenellaceae family, for its use in the prevention and/or treatment of renal insufficiency in humans or animals. It may be acute or chronic renal insufficiency, preferably chronic renal insufficiency.

Advantageously, such a bacterium, when it is administered to a human being or an animal exhibiting renal insufficiency, is capable of acting on the molecules produced in excess during this disease, such as in particular urinary CTGF, interleukin 18 of the renal tissue, plasma apolipoprotein A-IV, urinary CD14 mononuclear cells, serum renal insufficiency molecule-1, fibroblast growth factor-23, urinary liver fatty acid binding protein, urinary N-acetyl-bO glucosaminidase, neutrophil gelatinase associated with lipocalin, urinary retinol-binding protein 4, serum homocitrulline, serum symmetric dimethylarginine. In addition, it is a bacterium naturally present in the gut microbiota and whose administration does not cause side effects, unlike existing treatments.

Preferably, the bacteria for use according to the invention are administered within compositions. Thus, the invention also relates to compositions comprising at least one bacterium of the Christensenellaceae family for its use in the prevention and/or treatment of renal insufficiency in humans or animals.

Other features and advantages will become apparent from the detailed description of the invention which follows.

### DETAILED DESCRIPTION OF THE INVENTION

### Definitions

Within the meaning of the invention, “hyperproduction” or “excess production” of a molecule or of a marker means an excessive production of said molecule or of said marker compared to the production in a healthy person or animal without renal insufficiency.

Within the meaning of the invention, “marker” of a disease means a molecule or a substance whose assay makes it possible to follow the evolution of said disease.

### Bacteria According to the Invention

The subject of the invention is the use of at least one bacterium of the Christensenellaceae family for preventing and/or treating acute or chronic renal insufficiency, preferably chronic renal insufficiency, in humans or animals.

The invention therefore relates to a bacterium of the Christensenellaceae family for its use in preventing and/or treating renal insufficiency in humans or animals, in particular in humans or animals with renal insufficiency, in particular chronic, with hyperproduction of at least one marker chosen from: urinary CTGF, interleukin 18 of the renal tissue, beta-microglobulin, alpha1-microglobulin, microalbumin, plasma apolipoprotein A-IV, urinary CD14 mononuclear cells, clusterin, serum renal insufficiency molecule-1, fibroblast growth factor-23, urinary liver fatty acid binding protein, urinary N-acetyl-bO glucosaminidase, gelatinase neutrophil associated with lipocalin, osteopontin, urinary retinol-binding protein 4, serum homocitrulline, serum symmetric dimethylarginine.

Urinary CTGF (connective tissue growth factor) is an important profibrotic factor in kidney disease, the blockage of which increases kidney damage (Sánchez-López, E. et al. CTGF Promotes Inflammatory Cell Infiltration of the Renal Interstitium by Activating NF-B. J Am Soc Nephrol 20, 1513-1526 (2009)).

Interleukin 18 is a mediator of acute ischemic tubular necrosis and has been shown to be a rapid and reliable marker for the early detection of renal insufficiency, including acute renal insufficiency (Lin, X., Yuan, J., Zhao, Y. & Zha, Y. Urine interleukin-18 in prediction of acute kidney injury: a systemic review and meta-analysis. J. Nephrol. 28, 7-16 (2015)).

Alpha1-microglobulin is a marker of proximal tubular dysfunction, from the early phase of lesion when no histological damage is observed; microalbumin is a marker of tubular cell damage; beta-microglobulin is a marker excreted during tubular lesions; clusterin and molecule-1 are markers used in the early diagnosis of renal lesions and proximal tubular toxicity; urinary liver fatty acid binding proteins (L-FABP in the proximal tubule and H-FABP in the distal tubule) are markers of tubulointerstitial damage; urinary N-acetyl-bO glucosamine is a sensitive, persistent, and robust marker indicating tubular injury; neutrophil gelatinase is a marker of renal insufficiency when linked to inflammatory or infectious conditions; osteopontin is an overexpressed marker in renal biopsies from patients with hypertension_Cardenas-Gonzalez, M., Pavkovic, M. & Vaidya, V. S. Biomarkers of Acute Kidney Injury. in Comprehensive Toxicology: Third Edition 14-15, 147-163 (Elsevier Inc., 2017)).

Plasma apolipoprotein A-IV is a marker derived from intestinal enterocytes showing disease progression (Boes, E. et al. Apolipoprotein A-IV Predicts Progression of Chronic Kidney Disease: The Mild to Moderate Kidney Disease Study. J. Am. Soc. Nephrol. 17, 528-536 (2006)).

Urinary CD14 mononuclear cells are markers of renal insufficiency positively correlated with kidney volume and linked to polycystic kidney disease; fibroblast growth factor-23 is a marker of origin in osteocytes or osteoblasts, showing the progression of kidney disease and mortality; urinary retinol-binding protein 4 is a proximal tubule marker whose presence is linked to proximal tubule dysfunction (Lopez-Giacoman, S. Biomarkers in chronic kidney disease, from kidney function to kidney damage. World J. Nephrol. 4, 57 (2015)).

Serum homocitrulline is a byproduct of carbamylation and is therefore a marker of morbidity and mortality in renal insufficiency (Jaisson, S. et al. Homocitrulline as marker of protein carbamylation in hemodialyzed patients. Clin. Chem. Acta 460, 5-10 (2016)).

Serum symmetric dimethylarginine is a marker extracted from the renal artery showing endothelial damage, often related to hypertension in patients with renal insufficiency (Fleck, C., Schweitzer, F., Karge, E., Busch, M. & Stein, G. Serum concentrations of asymmetric (ADMA) and symmetric (SDMA) dimethylarginine in patients with chronic kidney diseases. Clin. Chem. Acta 336, 1-12 (2003) and Nijveldt, R. J. et al. Handling of asymmetric dimethylarginine and symmetrical dimethylarginine by the rat kidney under basal conditions and during endotoxaemia. Nephrol. Dial. Transplant. 18, 2542-2550 (2003)).

According to the invention, the bacteria of the Christensenellaceae family, when administered to a human being or an animal having renal insufficiency, in particular chronic renal insufficiency, are capable of acting on the molecules produced in excess during renal insufficiency, in particular during chronic renal insufficiency, in particular on the production of at least one marker chosen from: urinary CTGF, interleukin 18 of the renal tissue, beta-microglobulin, alpha1-microglobulin, microalbumin, plasma apolipoprotein A-IV, urinary CD14 mononuclear cells, clusterin, serum renal failure molecule-1, fibroblast growth factor-23, urinary liver fatty acid binding protein, urinary N-acetyl-bO glucosaminidase, neutrophil gelatinase associated with lipocalin, osteopontin, urinary retinol-binding protein 4, serum homocitrulline, serum symmetric dimethylarginine.

In renal insufficiencies, in particular chronic renal insufficiencies, presenting an increase in at least one marker chosen from: urinary CTGF, interleukin 18 of the renal tissue, beta-microglobulin, alphal-microglobulin, microalbumin, plasma apolipoprotein A-IV, urinary CD14 mononuclear cells, clusterin, serum renal insufficiency molecule-1, fibroblast growth factor-23, urinary liver fatty acid binding protein, urinary N-acetyl-bO glucosaminidase, gelatinase neutrophil associated with lipocalin, osteopontin, urinary retinol-binding protein 4, serum homocitrulline, serum symmetric dimethylarginine, their decrease is a sign of reduction of the renal cell degradation signaling pathways, that is to say, the renal cells at the origin of this production are less stimulated and protein carbamylation is less stimulated. From then on, the excessive degradation of the cells responsible for renal insufficiency, in particular chronic renal insufficiency, is slowed down and the system gradually returns to normal.

The useful bacteria according to the invention are administered to humans or animals in an amount effective for an action on at least one of these markers of chronic renal insufficiency, i.e., to reduce the production of at least one of these markers in the body.

According to a suitable embodiment, the bacterium or bacteria are administered at a dose of 10⁹ to 10¹² colony-forming units (CFU) per day, regardless of the weight of the person or animal. It will preferably be a single dose, i.e., administered once daily, or a dose before each meal (three times a day).

The useful bacterium or bacteria according to the invention are bacteria of the Christensenellaceae family, preferably of the genus Christensenella. It may be, in particular, Christensenella massiliensis, Christensenella timonensis and/or Christensenella minuta. According to a particularly suitable variant, it is Christensenella minuta.

These bacteria can be isolated from human stools for example according to the protocols published by Morotomi et al., 2012 (Morotomi, M., Nagai, F. & Watanabe, Y. Description of Christensenella minuta gen nov., nov. sp., Isolated from human faeces, which forms a separate branch in the order Clostridiales, and proposal of Christensenellaceae fam nov. INTERNATIONAL JOURNAL OF SYSTEMATIC AND EVOLUTIONARY MICROBIOLOGY 62, 144-149 (2012)) and NDongo et al., 2016 (Ndongo, S., Dubourg, G., Khelaifia, S., Fournier, P. E. & Raoult, D. Christensenella timonensis, a new bacterial species isolated from the human gut. New Microbes and New Infections 13, 32-33 (2016)). These documents also describe the culture methods of the bacteria that are useful according to the invention.

### Compositions

The useful bacteria according to the invention are preferably administered to the human or animal in a composition.

Thus, the invention also relates to a composition comprising at least one a bacterium of the Christensenellaceae family for its use in preventing and/or treating renal insufficiency, in particular chronic renal insufficiency, in humans or animals, in particular in humans or animals with hyperproduction of at least one marker chosen from: urinary CTGF, interleukin 18 of the renal tissue, beta-microglobulin, alpha1-microalbumin, plasma apolipoprotein A-IV, urinary CD14 mononuclear cells, clusterin, serum renal insufficiency molecule-1, fibroblast growth factor-23, urinary liver fatty acid binding protein, urinary N-acetyl-bO glucosaminidase, gelatinase neutrophil associated with lipocalin, osteopontin, urinary retinol-binding protein 4, serum homocitrulline, serum symmetric dimethylarginine.

The bacterium or bacteria are present in an effective amount in the composition, allowing an effect on the renal insufficiency, in particular chronic renal insufficiency, of the treated persons or animals.

Preferably, the useful composition according to the invention comprises 10⁶ to 10¹² colony-forming units (CFU) of bacteria of the Christensenellaceae family per daily dose of composition to be administered. Preferably, this corresponds to a daily dose of bacteria to be administered, regardless of the weight of the person or the animal. Preferably, this daily dose is administered once per day.

The useful composition according to the invention may be in liquid form. It may in particular comprise bacteria of the Christensenellaceae family and a culture medium for said bacteria that makes it possible to preserve them, such as, for example, Columbia anaerobic medium enriched with sheep blood, or an equivalent medium not containing an animal byproduct.

When the compositions are in liquid form, they are preferably frozen, maintained at −20° C. in a sealed bag.

According to one variant, the useful composition according to the invention may be in solid form. In this case, the bacteria may be present in freeze-dried form, and the compositions may also comprise excipients such as, for example, microcrystalline cellulose, lactose, sucrose, fructose, levulose, starches, stachyose, raffinose, amylum, calcium lactate, magnesium sulphate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, sorbitol and/or glycerol.

The useful compositions according to the invention may in particular be in the form of powder, microencapsulated powder, gelcap, capsule, tablet, lozenge, granules, emulsion, suspension or suppository. According to a particularly suitable embodiment, they may be in a gastro-resistant form, such as a coated tablet containing microencapsulated bacteria.

When the compositions are in solid form, they are preferably packaged in capsules or in a coating hermetically sealed against light and oxygen maintained at an ambient temperature of between 15° C. and 40° C. and a humidity level between 3% and 70%.

The bacteria can be used alive, or inactivated, for example by heat, exposure to an appropriate pH, gamma radiation or high pressure.

They can all be alive or all inactivated.

Preferably, at least part of the bacteria are alive, in particular at least 50% (by number), even more preferably at least 90% (by number).

Thus, according to a suitable embodiment, the bacteria present in the useful composition according to the invention are at least 50% living bacteria (by number), preferably at least 90% living bacteria (by number), and even more preferentially all living.

The useful bacteria according to the invention, and in particular the compositions that include it, can be administered orally, topically, through the lungs (inhalation) or rectally.

The useful compositions according to the invention, in addition to the useful bacteria according to the invention, can comprise other compounds, such as:
at least one probiotic, and/or
at least one bacterium producing lactic acid, which makes it possible to create an anaerobic environment favorable to Christensenellaceae, such as at least one bacterium chosen from bacteria of the genus Lactobacillus spp., Bifidobacterium spp., Streptococcus spp. and/or at least one other organism promoting the anaerobic conditions necessary for the survival of Christensenellaceae, such as at least one yeast chosen from Saccharomyces spp. or microorganisms of the Methanobacteriaceae family, and/or
at least one bacterium associated with the Christensenellaceae ecosystem, since the latter facilitate the survival of said bacteria in the intestine, such as at least one bacterium chosen from bacteria of the phylum Firmicutes, Bacteroidetes, Actinobacteria, Tenericutes, and Verrucomicrobia, and/or
at least one bacterium chosen from bacteria of the order Clostridales, Verrucomicrobiales, Aeromonadales, Alteromonadales, ML615J-28, RF32, YS2, of the Clostridiaceae, Lachnospiraceae, Ruminococcaceae, Bacteroidaceae, Enterococcaceae, Rikenellaceae, Dehalobacteriaceae, Veillonellaceae family, and/or
at least one bacterium chosen from bacteria of the genus Faecalibacterium, Akkermansia, Eubacterium and Oscillospira such as for example Faecalibacterium prausnitzii, Akkermansia muciniphila, Eubacterium halii, Oscillospira guilliermondii, and/or
at least one prebiotic such as for example at least one prebiotic chosen from galactooligosaccharides, fructooligosaccharides, inulins, arabinoxylans, beta-glucans, lactoglobulins and/or beta-caseins, and/or
at least one polyphenol such as for example at least one polyphenol chosen from quercetin, kaempferol, resveratrol, flavones (such as luteolin), flavan-3-ols (such as catechins), flavanones (such as naringenin), isoflavones, anthocyanidins, proanthocyanidins, and/or
at least one mineral and/or at least one vitamin and/or at least one nutritional agent, and/or
at least one pharmaceutical active principle having an effect in preventing and/or treating renal insufficiency, such as for example vitamin D, sevelamer, sodium polystyrene sulfonate, calcium, a calcimimetic agent (cinacalcet, for example), an antihypertensive, a diuretic, a statin, iron and/or an erythropoietin derivative.

The invention is now illustrated by examples of useful bacteria according to the invention, methods of culturing these bacteria, examples of compositions containing them and test results demonstrating the effectiveness of the bacteria of the Christensenellaceae family on renal insufficiency, in particular on chronic renal insufficiency.

### EXAMPLES

### Example 1: Christensenella minuta

The Christensenella minuta bacteria can be cultured according to the operating protocol described below.

1/ Dissolve a dehydrated RCM (“Reinforced Clostridial Medium”) in distilled water

2/ Add 0.5 mL/L of resazurin-Na solution (0.1% w/v)

3/ Bring to a boil and cool to room temperature while injecting a gaseous mixture of 80% N₂ and 20% CO₂

4/ Spread the medium under the same gaseous atmosphere in anoxic Hungate-type tubes or in serum vials, then autoclave

5/ Before use, add 1.0 g of sodium carbonate per liter from a sterile anoxic stock solution prepared with a gaseous mixture of 80% N₂ and 20% CO₂

6/ Check the pH of the medium after autoclaving and adjust the pH between 7.3 and 7.5, using a sterile anoxic stock solution of sodium bicarbonate (5% w/v) prepared in a gaseous atmosphere at 80% N₂ and at 20% CO₂.

### Example 2: Christensenella massiliensis

The Christensenella massiliensis bacteria can be cultured according to the operating protocol described below.

1/ Prepare a carboxymethylcellulose (N₂/CO₂) medium by following the instructions below provided by DSMZ (Deutsche Sammlung von Mikroorganismen and Zell-kulturen), presented in Table 1.

TABLE 1

| | | |
|---|---|---|
| Casitone | 30.0 | g |
| Yeast extract | 5.0 | g |
| K₂HPO₄ | 5.0 | g |
| Na-resazurin solution (0.1% w/v) | 0.5 | mL |
| L-Cysteine-HCl × H2O | 0.5 | g |
| D-Glucose | 4.0 | g |
| Cellobiose | 1.0 | g |
| Maltose | 1.0 | g |
| Na₂CO₃ | 1.0 | g |
| Meat filtrate (see Table 2) | 1000 | mL |

2/ Dissolve the different constituents listed in the table above, except cysteine, carbohydrates and carbonate.

3/ Boil the medium for 1 min, then let it cool to room temperature under a gaseous atmosphere containing 80% N₂ and 20% CO₂.

4/ Add 0.5 g/L of L-cysteine-HCl×H₂O and pour it under the same gaseous atmosphere into Hungate-type tubes (for strains requiring meat particles, introduce these first in the tube; use 1 part meat particles to 4 or 5 parts liquid).

5/ Autoclave at 121° C. for 20 min.

6/ After autoclaving, add glucose, cellobiose, maltose and starch from sterile anoxic stock solutions prepared with 100% N₂ gas and carbonate from a sterile anoxic stock solution prepared under gaseous mixtures at 80% N₂ and 20% CO₂.

7/ Adjust the pH of the medium to 7, if necessary.

The composition of the meat filtrate is shown in Table 2.

TABLE 2

| | | |
|---|---|---|
| Ground meat (no fat) | 500.0 | g |
| 1N NaOH | 25.0 | mL |
| Distilled water | 1000 | mL |

The meat filtrate is prepared as follows.

a/ Use lean beef or horse meat.

b/ Remove fat and connective tissue before chopping.

c/ Mix the meat, water and NaOH, then boil for 15 minutes with stirring.

d/ Allow to cool to room temperature, remove fat from the surface and filter, retaining meat particles and filtrate.

e/ Add water to the filtrate to a final volume of 1000.0 mL.

The bacteria must be grown under anaerobic conditions at 37° C.

### Example 3: Christensenella timonensis

The Christensenella timonensis bacteria can be cultivated according to the same procedure as that described in Example 2 for Christensenella massiliensis.

### Example 4: Useful Composition According to the Invention in Liquid Form

An example of a useful composition according to the invention in liquid form is a composition comprising Christensenella minuta 10⁹ CFU/mL in the RCM anaerobic culture medium described above, modified to contain no product of animal origin and enriched with 5% glycerol.

The composition of Example 4 was obtained from an RCB (“research cell bank”) prepared with Christensenella minuta 10¹⁰ CFU/mL and stored frozen at −20° C. in a bag hermetically sealed to oxygen.

The frozen composition must be warmed to room temperature until a liquid form is found before use.

### Example 5: Useful Composition According to the Invention in Solid Form

An example of a useful composition according to the invention in freeze-dried form can be obtained by lyophilization of the composition of Example 4 in the frozen state.

### Trials Demonstrating the Efficacy of the Invention in Preventing and Treating Renal Insufficiency.

### Study of the Effect of the Invention in Treating Renal Insufficiency in Vitro

The objective of this study is to demonstrate in vitro the effect of bacteria of the Christensenellaceae family in the treatment of renal insufficiency. The demonstration was carried out on two of the markers of renal insufficiency, in particular of chronic renal insufficiency: homocitrulline and serum symmetric dimethylarginine.

Carbamylation is a non-enzymatic post-translational modification of proteins that is characterized by the binding of isocyanic acid to protein amino groups (α-NH2 or ε-NH2). This reaction leads to the formation of carbamylation derivatives (CDP), the most representative being homocitrulline (HCit), which is generated by the binding of isocyanic acid to the ε-NH2 group of the side chain of lysine residues.

Homocitrulline is a carbamylation derivative that has been identified as a major marker of morbidity and mortality in patients with renal insufficiency, particularly chronic renal insufficiency. Homocitrulline can be absorbed in the intestine.

The procedure of the study is described below.

1/ Fermentation protocol using human feces containing Christensenella spp.:
The donors must not have taken antibiotics during the six months preceding the experiment and have no history of gastrointestinal disorders. The donors were between 18 and 60 years old.
The collection of fresh samples of their feces is obtained in sterile plastic containers, stored in anaerobic bottles containing a 2.5 L sachet of AnaeroGen™ from Oxoid™ (O₂<0.1%; CO₂: 7-15%). These samples were brought to the laboratory within two hours of their production.
Feces samples were diluted 1/5 (weight/volume) in phosphate-buffered saline (1M) (PBS), pH 7.4. The suspension was homogenized in a stomacher for 120 seconds.
Basic nutrient medium: the basic nutrient medium was prepared from 2 g/L tryptone soy broth, 2 g/L yeast extract, 0.1 g/L NaCl, 0.04 g/L K₂HPO₄, 0.01 g/L MgSO₃.7H2O, 0.01 g/L CaCl₂.6H2O, 2 g/L NaHCO₃, 0.5 g/L L-cystine HCl, 2 mL/L tween 80, 10 μL/L vitamin K1, 0.05 g/L heme, 0.05 g/L bile salts, 4 mL/L resazarin (pH 7)
Fermentation in a bio fermenter: The 20 mL-capacity bio fermenters contained 18 mL of autoclaved base nutrient medium (121° C. for 15 minutes) poured aseptically into the sterile bio fermenters. This system was allowed to stand overnight with oxygen-free nitrogen bubbling through the medium at a rate of 2 mL/min. The pH was maintained between 6.7 and 6.9 using HCl or NaOH (0.5 M). The temperature of each bio fermenter was controlled at 37° C. and the contents of the container were homogenized with a magnetic mixer
a mixture of predigested proteins (0.35 g) was added to the containers before inoculation with 2 mL of fecal inocula at T0. The predigested proteins were obtained according to the gastrointestinal digestion protocol adapted from that of Versantvoort et al (2005).
the samples were collected before fermentation (T0) and after 48 hours of fermentation (T48), and frozen at −80° C. until analysis.

2/ Quantification of homocitrulline and serum symmetric dimethylarginine:
50 μL of samples collected and stored at −80° C. was mixed with 20 μL of MilliQ water containing internal standards.
The mixture was mixed and filtered through a 5-kDa threshold filter to remove macromolecules.
The metabolites were detected by capillary electrophoresis-time-of-flight mass spectrometry (CE-TOFMS) analyses. The peak detection limit was determined based on the signal-to-noise ratio, S/N=3.

Relative peak area=(metabolite peak area)/(internal standard peak area×amount of sample).

3/ Quantification of Christensenella spp.
The DNA contained in the samples was extracted using the NucleoSpin®96 Soil kit from Macherey-Nagel according to the manufacturer's instructions.
The total extracted DNA was then randomly fragmented into 350 bp fragments and then used to build a library using the NEBNext Ultra II kit by New England Biolabs according to the manufacturer's instructions.
The library was then sequenced using 2×150 bp pairwise sequencing on an Illumina HiSeq platform.
The abundance of bacteria was measured by creating a metagenomic species catalog (MGS) from a reference catalog containing 22M genes. These MGSs were then associated with an appropriate taxonomic level. In the case of Christensenella, these were detected at the genus level and are therefore referred to in this experiment as Christensenella spp.

The relative amount of symmetric homocitrulline and dimethylarginine and the relative abundance of Christensenella spp. were analyzed and correlated, obtaining a linear regression of R=−0.45 (n=18).

The results are shown in Table 3.

TABLE 3

| | Relative | Relative amount of | |
|---|---|---|---|
| | abundance of | serum symmetric | Relative amount of |
| | Christensenella | dimethylarginine | homocitrulline |
| Samples | spp (×10⁻²) | (×10⁻⁵) | (×10⁻⁵) |
| V1 | 7.55 | 0 | 2.87 |
| V2 | 3.18 | 0 | 0 |
| V3 | 8.19 | 0 | 4.18 |
| V4 | 2.63 | 0 | 3.50 |
| V5 | 1.26 | 0 | 2.92 |
| V6 | 2.87 | 0 | 3.47 |
| V7 | 7.20 | 3.78 | 4.34 |
| V8 | 2.91 | 4.21 | 18.82 |
| V9 | 6.32 | 0 | 0 |
| V10 | 1.21 | 5.10 | 39.15 |
| V11 | 4.23 | 5.92 | 19.14 |
| V12 | 1.49 | 4.61 | 14.81 |
| V13 | 9.83 | 0 | 0 |
| V14 | 4.12 | 0 | 6.14 |
| V15 | 6.45 | 0 | 0 |
| V16 | 2.02 | 0 | 7.56 |
| V17 | 5.23 | 0 | 11.77 |
| V18 | 7.57 | 0 | 7.68 |

There is a negative correlation between bacteria of the Christensenellaceae family and homocitrulline and serum symmetric dimethylarginine, which demonstrates a protective effect of the bacteria of the Christensenellaceae family against renal insufficiency.

Thus, the bacteria of the Christensenellaceae family are able to act by reducing the production of markers of renal insufficiency, in particular homocitrulline and serum symmetric dimethylarginine. They can therefore be used to prevent and/or treat renal insufficiency, whether acute or chronic.

1. A bacterium of the Christensenellaceae family for its use in preventing and/or treating renal insufficiency in humans or animals.

2. The bacterium of the Christensenellaceae family for its use according to claim 1, in human beings or animals exhibiting renal insufficiency correlated with hyperproduction of homocitrulline and/or of serum symmetric dimethylarginine.

3. The bacterium of the Christensenellaceae family for its use according to claim 1, Christensenellaceae for its use in preventing and/or treating chronic renal insufficiency in humans or animals.

4. The bacterium of the Christensenellaceae family for its use according to claim 1, characterized in that said bacterium is a bacterium of the genus Christensenella.

5. The bacterium of the Christensenellaceae family for its use according to claim 1, characterized in that said bacterium is selected from Christensenella massiliensis, Christensenella timonensis and Christensenella minuta.

6. A composition comprising at least one bacterium of the Christensenellaceae family for its use in preventing and/or treating renal insufficiency in humans or animals.

7. The composition for its use according to claim 6, in the prevention and/or treatment of chronic renal insufficiency in humans or animals.

8. The composition for its use according to claim 6, characterized in that it is in liquid form.

9. The composition for its use according to claim 6, characterized in that it is in solid form.

10. The composition for its use according to claim 9, characterized in that the bacteria are present in freeze-dried form.

11. The composition for its use according to claim 6, characterized in that the bacteria present are at least 50% living bacteria (by number).

12. The composition for its use according to claim 6, characterized in that the bacteria present are at least 90% living bacteria (by number).

13. The composition for its use according to claim 6, orally, rectally or inhaled.

14. The composition for its use according to claim 6, characterized in that it is in the form of powder, microencapsulated powder, gelcap, capsule, tablet, lozenge, granules, emulsion, suspension or suppository.

15. The composition for its use according to claim 6, characterized in that it is in a gastro-resistant form.

16. The composition for its use according to claim 6, characterized in that it comprises at least one probiotic and/or at least one prebiotic.

17. The composition for its use according to claim 6, characterized in that it also comprises:
at least one bacterium producing lactic acid and/or at least one other organism promoting the anaerobic conditions necessary for the survival of Christensenellaceae, and/or
at least one bacterium associated with the Christensenellaceae ecosystem, and/or
at least one bacterium chosen from bacteria of the genus Faecalibacterium, Akkermansia, Eubacterium and Oscillospira, and/or
at least one polyphenol, and/or
at least one mineral and/or at least one vitamin and/or at least one nutritional agent, and/or
at least one pharmaceutical active principle having an effect of preventing or treating renal insufficiency.
